(19) United States
(12) Patent Application Publication
(10) Pub. No.: US 2023/0210795 A1
(43) Pub. Date: Jul. 6, 2023

(54) 3-HYDROXYBUTYRIC-ACID-CONTAINING OIL AND FAT COMPOSITION

(71) Applicant: OSAKA GAS CO., LTD., Osaka-shi, Osaka (JP)

(72) Inventors: Masayuki SUGIMOTO, Osaka-shi, Osaka (JP); Jun TSUBOTA, Osaka-shi, Osaka (JP); Shin ARIMOTO, Tokyo (JP)

(21) Appl. No.: 17/928,003

(22) PCT Filed: May 19, 2021
(86) PCT No.: PCT/JP2021/018957

§ 371 Date: Nov. 28, 2022

(30) Foreign Application Priority Data
May 28, 2020 (JP) 2020-093000

(51) Int. Cl. A61K 31/19; A61K 9/16

(52) U.S. Cl. CPC A61K 31/19; A61K 9/1617

(57) ABSTRACT

A 3-hydroxybutyric-acid-containing fat and/or oil composition that contains 3-hydroxybutyric acid and/or a salt thereof and that contains fat and/or oil particles, the fat and/or oil particles having a plate-like shape and containing a fat and/or oil component that includes at least one type of XXX triglyceride having a Cₓ fatty acid residue X at positions 1 to 3 of glycerin, x, which represents the number of carbon atoms, being an integer selected from 10 to 22, and the fat and/or oil component comprising a β-form fat and/or oil.

### TECHNICAL FIELD

The present invention relates to a fat and/or oil composition containing 3-hydroxybutyric acid.

### BACKGROUND ART

3-Hydroxybutyric acid (hereinafter also simply referred to as “3HB”) is a substance that is originally present in the human body, and is attracting attention as a breakthrough energy source that can replace carbohydrates.

For example, when a medium-chain fatty acid (MCT) contained in coconut oil etc. is ingested, 3HB is then metabolized in the body and transported through the bloodstream, and converted into energy. This process can convert 3HB into energy more quickly than the metabolism of carbohydrates via glycolysis.

Further, 3HB has an effect of inhibiting cells from absorbing fat and sugar. In addition to this, 3HB is also considered to have ameliorating effects on cognitive function and long-term continuous memory function, and have preventive effects on Alzheimer's disease.

In view of such functions of 3HB, the application of 3HB as an energy substance for athletes or diet and health foods has been studied.

However, 3HB and salts thereof (in particular, potassium salt of 3HB), which have high water solubility and deliquescence, are extremely difficult to handle in a solid state, such as powder.

In addition to this, 3HB has a strong sour taste, which causes discomfort when orally ingested. In order to reduce such a strong sour taste, a method of neutralizing 3HB into a salt of 3HB and using the salt of 3HB can also be considered. However, this method results in excessive salt intake, which is not preferable. A method of adding an anticaking agent, such as silicon dioxide, calcium silicate, or dextrin, can also be considered. However, since such compounds may have an upper intake limit or contain carbohydrates, their use in combination with 3HB is not preferable.

Thus, there is a need for a method for improving the handling of 3HB in a solid state and reducing sour taste in oral intake of 3HB.

### SUMMARY OF INVENTION

### Technical Problem

In view of the above circumstances, an object of the present invention is to provide a fat and/or oil composition containing 3-hydroxybutyric acid, the composition being less deliquescent and having a reduced sour taste, and being easy to handle in the form of a powder.

### Solution to Problem

As a result of extensive research to achieve the above object, the present inventors found that when a composition comprising 3HB and specific fat and/or oil particles is produced, the deliquescence and sour taste of 3HB can both be suppressed. The inventors have accomplished the present invention as a result of further research based on this finding.

More specifically, the present invention provides the following fat and/or and oil compositions containing 3-hydroxybutyric acid.

### Item 1.

A 3-hydroxybutyric-acid-containing fat and/or oil composition comprising

3-hydroxybutyric acid and/or a salt of 3-hydroxybutyric acid, and

fat and/or oil particles,
the fat and/or oil particles having a plate-like shape and comprising a fat and/or oil component containing at least one type of XXX triglyceride having a Cₓ fatty acid residue X at positions 1 to 3 of glycerine, wherein x, which represents the number of carbon atoms, is an integer selected from 10 to 22, and
the fat and/or oil component comprising β-form fat and/or oil.

### Item 2.

The composition according to Item 1, wherein the fat and/or oil particles are present in an amount of 0.5 to 100 parts by mass per 100 parts by mass of the total of the 3-hydroxybutyric acid and/or the salt of 3-hydroxybutyric acid.

### Item 3.

The composition according to Item 1 or 2, wherein the salt of 3-hydroxybutyric acid is at least one member selected from the group consisting of sodium salt, calcium salt, and magnesium salt, and potassium salt.

### Item 4.

The composition according to any one of Items 1 to 3, wherein the 3-hydroxybutyric acid and/or the salt of 3-hydroxybutyric acid has an R configuration.

### Item 5.

A method for improving the deliquescence of 3-hydroxybutyric acid and/or a salt of 3-hydroxybutyric acid,

the method comprising mixing the 3-hydroxybutyric acid and/or the salt of 3-hydroxybutyric acid with fat and/or oil particles,

the fat and/or oil particles having a plate-like shape and containing at least one type of XXX triglyceride having a Cₓ fatty acid residue X at positions 1 to 3 of glycerin, wherein x, which represents the number of carbon atoms, is an integer selected from 10 to 22, and

the fat and/or oil component comprising B-form fat and/or oil.

### Item 6.

A method for reducing the sour taste of 3-hydroxybutyric acid and/or a salt of 3-hydroxybutyric acid,

the method comprising mixing the 3-hydroxybutyric acid and/or the salt of 3-hydroxybutyric acid with fat and/or oil particles,

the fat and/oil particles having a plate-like shape and comprising a fat and/or oil component containing at least one type of XXX triglyceride having a Cₓ fatty acid residue X at positions 1 to 3 of glycerin, wherein x, which represents the number of carbon atoms, is an integer selected from 10 to 22, and

the fat and/or oil component comprising B-form fat and/or oil.

### Advantageous Effects of Invention

The fat and/or oil composition containing 3-hydroxybutyric acid according to the present invention is less deliquescent and has reduced sour taste, and is easy to handle as a powder.

### BRIEF DESCRIPTION OF DRAWINGS

FIG. 1 is a graph showing the FT-IR measurement results of a fat and/or oil composition comprising 3-hydroxybutyric acid according to the present invention.

FIG. 2 is a photograph showing the appearance of the crystal composition (B-form fat and/or oil) obtained in Production Example 7 of the present invention.

FIG. 3 is a photograph showing the appearance of the crystal composition (β-form fat and/or oil) of the present invention obtained in Production Example 7.

FIG. 4 is a photograph of the solid (α-form fat and/or oil) obtained in Production Comparative Example 3 of the present invention.

FIG. 5 is a micrograph of the crystalline composition (B-form fat and/or oil) of the present invention obtained in Production Example 7.

FIG. 6 is a micrograph of the solid (α-form fat and/or oil) obtained in Comparative Example 3 of the present invention.

FIG. 7 is an X-ray diffraction diagram of the crystalline composition (B-form fat and/or oil) of the present invention obtained in Production Example 7.

FIG. 8 is an X-ray diffraction diagram of the solidified product (α-form fat and/or oil) obtained in Comparative Example 3 of the present invention.

FIG. 9 is a photograph of the appearance of the composition of the present invention obtained in Example 1.

FIG. 10 is a photograph of the appearance of the composition obtained in Comparative Example 1 of the present invention.

FIG. 11 is a photograph of the appearance of the composition of the present invention obtained in Example 6.

FIG. 12 is a photograph of the appearance of the composition obtained in Comparative Example 4 of the present invention.

### DESCRIPTION OF EMBODIMENTS

1) Fat and/or oil composition comprising 3-Hydroxybutyric Acid

The fat and/or oil composition containing 3-hydroxybutyric acid of the present invention (which may be simply referred to below as “3HB-containing composition”) comprises 3HB and/or a salt of 3HB and fat and/or oil particles.

1.1) 3-Hydroxybutyric Acid and/or Salt of 3-Hydroxybutyric Acid

3-Hydroxybutyric acid (3HB) is a compound represented by the following formula:

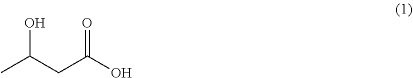

3HB can be in the form of a salt. Examples of salts of 3HB include, but are not limited to, sodium salt, potassium salt, lithium salt, magnesium salt, calcium salt, ammonium salt, and dicyclohexylammonium salt of 3HB. These salts can be used in a combination.

Examples of the method for producing 3HB used in the present invention include, but are not limited, to known methods. The same applies to the production of salts of 3-hydroxybutyric acid. The salts can be produced by commonly used processes, such as salt formation, desalting, and salt exchange.

The 3HB used in the present invention can be in the form of a liquid (e.g., an aqueous solution or an ethanol solution) or a solid, and is particularly preferably in the form of a solid. 3HB in such a form tends to have an increased effect of suppressing deliquescence of the obtained composition. When the composition is prepared from a liquid, the 3HB content of the composition may be increased by removing water or a solvent that remains in the composition by using an evaporator after or during mixing, or by adding seed crystals to promote crystallization.

The size of 3HB is not limited, and can be appropriately set according to the type of salt etc. of the 3HB salt.

1.2) Fat and/or oil particles

The fat and/or oil particles contained in the 3HB-containing composition of the present invention have a plate-like shape and contain a fat and/or oil component comprising at least one type of XXX triglyceride having a Cₓ fatty acid residue X at positions 1 to 3 of glycerin, wherein x, which represents the number of carbon atoms, is an integer selected from 10 to 22, and the fat and/or oil component comprises B-form fat and/or oil.

The fat and/or oil particles are preferably in the form of a powder solid at ordinary temperature (20° C.).

The fat and/or oil particles preferably have a loose bulk density of 0.05 to 0.6 g/cm³, more preferably 0.1 to 0.4 g/cm³, and even more preferably 0.1 to 0.3 g/cm³.

The term “loose bulk density” as used herein means a packing density determined after letting a powder fall naturally.

The loose bulk density (g/cm³) can be obtained, for example, by: letting an appropriate amount of fat and/or oil particles fall approximately 2 cm above the upper-opening end of a 25-mL graduated cylinder having an inner diameter of 15 mm to loosely fill the graduated cylinder with the fat and/or oil particles; measuring the mass (g) of the fat and/or oil particles filled in the cylinder, and reading the volume (mL) of the fat and/or oil particles filled in the cylinder; and calculating the mass (g) of the fat and/or oil particles per cm³.

Preferably, the measurement is performed three times, and the average of the three measurements is taken.

The fat and/or oil particles usually have a plate-like shape, and preferably have an average particle diameter (effective diameter) of, for example, 0.5 to 200 μm, more preferably 1 to 100 μm, even more preferably 1 to 60 μm, and particularly preferably 1 to 30 μm.

Here, the average particle diameter (effective diameter) in the present invention is a value determined by wet measurement based on laser diffraction/scattering methods (IS0133201 and IS09276-1) using a particle size distribution analyzer (e.g., instrument name: Microtrac MT3300ExII, produced by Nikkiso Co., Ltd.) (d50: a measured value of particle diameter at 50% in the cumulative particle size distribution).

The term “effective diameter” means a diameter of a spherical particle used to fit a measured diffraction pattern of the measurement-target particle crystal to a theoretical diffraction pattern obtained under the assumption that the measurement target particle is spherical.

In this way, in the laser diffraction/scattering methods, an effective diameter is calculated by fitting a measured diffraction pattern to a theoretical diffraction pattern obtained under the assumption that the measurement target particle is spherical. For this reason, even when the measurement target particle has a plate-like shape or a spherical shape, the measurement can be performed according to the same principle. Herein, the plate-like particle preferably has an aspect ratio of 1.1 or more, more preferably 1.2 to 3.0, even more preferably 1.3 to 2.5, and particularly preferably 1.4 to 2.0.

The term “aspect ratio” as used herein is defined as the ratio of the length of a longer side to the length of a shorter side of a rectangle circumscribing a particle figure in such a manner that the area enclosed by the rectangle is minimized. When the particle has a spherical shape, the aspect ratio is smaller than 1.1. In a conventional method in which a fat and/or oil having a high solid fat content at normal temperature, such as a highly hydrogenated oil, is melted and directly sprayed, fat and/or oil particles have a spherical shape due to the surface tension, and have an aspect ratio of less than 1.1. The aspect ratio can be determined by directly observing arbitrarily selected particles with, for example, an optical microscope or a scanning electron microscope to measure the lengths in the major-axis and minor-axis directions of each particle and calculating the average aspect ratio of the measured particles.

1.2.1) Fat and/or Oil Component

The fat and/or oil particles preferably contain a fat and/or oil component. The fat and/or oil component preferably contains at least one type of XXX triglyceride, and may optionally further contain other triglycerides.

The fat and/or oil component preferably contains β-form fat and/or oil. The β-form fat and/or oil as referred to herein means a fat and/or oil consisting only of β-form crystals, i.e., one of the polymorphs of fat and/or oil crystals. Fats and/or oils of other polymorphs include β′-form fat and/or oil and α-form fat and/or oil. β′-form fat and/or oil means a fat and/or oil consisting only of β′-form crystals, i.e., one of the polymorphs of fat and/or oil crystals. α-form fat and/or oil means a fat and/or oil consisting only of α-form crystals, i.e., one of the polymorphs of fat and/or oil crystals. Some fat and/or oil crystals are identical in composition but have a different sub-lattice structure (crystal structure); these are called crystal polymorphs. Typical examples include hexagonal packing, orthorhombic perpendicular packing, and triclinic parallel packing, which are respectively referred to as α-form, β′-form, and β-form.

The melting points of the polymorphs increase in the order of the α-form, β′-form, and β-form. The melting points of the polymorphs vary depending on the type of Cₓ fatty acid residue X. Accordingly, Table 1 below shows the melting points (° C.) of polymorphs when the Cₓ fatty acid residue X is tricaprin, trilaurin, trimyristin, tripalmitin, tristearin, triarachidin, and tribehenin. Table 1 was prepared based on Nissim Garti et al., “Crystallization and Polymorphism of Fats and Fatty Acids,” Marcel Dekker Inc., 1988, pp. 32-33. In preparing Table 1, each melting point temperature (° C.) was rounded to the nearest whole number. When the composition of fat and/or oil and melting points of polymorphs of the fat and/or oil are known, it is at the very least possible to detect whether the fat and/or oil contains B-form fat and/or oil.

TABLE 1

| | α-resin (° C.) | β′-resin (° C.) | β-resin (° C.) |
|---|---|---|---|
| Tricaprin | −9 | 16 | 32 |
| Trilaurin | 15 | 34 | 47 |
| Trimyristin | 33 | 45 | 59 |
| Tripalmitin | 45 | 57 | 66 |
| Tristearin | 55 | 63 | 74 |
| Triarachidin | 62 | 69 | 78 |
| Tribehenin | 68 | 74 | 83 |

General methods for identifying these polymorphs include the X-ray diffraction method. The diffraction conditions are given by the following Bragg's equation:

2dsinθ=nλ(wherein n=1,2,3, . . . )

A diffraction peak appears at a position satisfying this equation. In the equation, d represents a lattice constant, θ represents a diffraction (incident) angle, λ represents a wavelength of X-ray, and n represents a natural number. From a diffraction peak at 2θ=16 to 27° corresponding to a short spacing, information on packing (sub-lattice) in the crystal side surface is obtained, and the polymorph can be identified. In particular, in the case of triacylglycerol, characteristic peaks of the β-polymorph appear at 2θ=near 19°, near 23°, near 24° (near 4.6 Å, near 3.9 Å, near 3.8 Å), and characteristic peak of the α-polymorph appears near 21° (4.2 Å). Each peak may have an error of ±0.5°. The X-ray diffraction measurement is performed, for example, using an X-ray diffractometer (Ultima IV, a horizontal sample mount X-ray diffractometer, produced by Rigaku Corporation) maintained at 20° C. As the X-ray light source, CuKα radiation (1.54 Å) is most frequently used.

Further, the crystal polymorphism of the fat and/or oil can also be predicted by differential scanning calorimetry (DSC). For example, the β-form fat and/or oil can be predicted by predicting the crystal structure of the fat and/or oil based on the DSC curve obtained by differential scanning calorimetry (product number: BSC6220, produced by SII NanoTechnology Inc.) at a temperature increase rate of 10° C./min up to 100° C.

The fat and/or oil component preferably comprises B-form fat and/or oil, or contains β-form fat and/or oil as a main component (more than 50 mass %). In a preferred embodiment, the fat and/or oil substantially consists of β-form fat and/or oil.

In a more preferable embodiment, the fat and/or oil component is composed of β-form fat and/or oil. In a particularly preferred embodiment, the fat and/or oil component consists only of β-form fat and/or oil. The case where the fat and/or oil component consists only of β-form fat and/or oil refers to a case where neither α-form fat and/or oil nor β′-form fat and/or oil are detected by differential scanning calorimetry. According to another preferable embodiment, in an X-ray diffraction measurement, the fat and/or oil component has a diffraction peak near 4.5 to 4.7 Å, and preferably near 4.6 Å, but has no X-ray diffraction peak of short spacing of the α-fat and/or oil or β′-form fat and/or oil in Table 1; in particular, it has no diffraction peak near 4.2 Å. In such a case as well, it can be determined that the entire fat and/or oil component is β-form fat and/or oil. In still another embodiment of the present invention, the fat and/or oil component preferably consists only of β-form fat and/or oil; however, it may contain other forms of fats and/or oils, such as α-form fat and/or oil and β′-form fat and/or oil. Here, “containing β-form fat and/or oil” in the fat and/or oil component of the present invention or an index for the relative quantity ratio of β-form fat and/or oil to the total amount of α-form fat and/or oil+β-form fat and/or oil can be speculated from the intensity ratio of the characteristic peak of β-form fat and/or oil and the characteristic peak of α-form fat and/or oil among X-ray diffraction peaks, i.e., [characteristic peak intensity of β-form fat and/or oil/(characteristic peak intensity of α-form fat and/or oil+characteristic peak intensity of β-form fat and/or oil)] (which may be also referred to below as the peak intensity ratio). Specifically, based on the above findings of the X-ray diffraction measurement, the proportions of a peak intensity at 20=19° (4.6 Å), which is the characteristic peak of β-form fat and/or oil, and a peak intensity at 20=21° (4.2 Å), which is the characteristic peak of α-form fat and/or oil, i.e., 19°/(19°+21°) [4.6 Å/(4.6 Å+4.2 Å)], are calculated as an index that indicates the amount of β-form fat and/or oil present in the fat and/or oil component, and “containing β-form fat and/or oil” can be understood therefrom. In the present invention, it is preferable that the entire fat and/or oil component is β-form fat and/or oil (that is, the peak intensity ratio=1). The lower limit of the peak intensity ratio is, for example, 0.4 or more, preferably 0.5 or more, more preferably 0.6 or more, even more preferably 0.7 or more, particularly preferably 0.75 or more, and particularly more preferably 0.8 or more. When the peak intensity is 0.4 or more, β-form fat and/or oil can be regarded as being present as the main component (more than 50 mass %). The upper limit of the peak intensity ratio is preferably 1; however, the upper limit can be, for example, 0.99 or less, 0.98 or less, 0.95 or less, 0.93 or less, 0.90 or less, 0.85 or less, or 0.80 or less. The peak intensity ratio can be any one of the above-mentioned lower- and upper-limit values, or any combination thereof.

### 1.2.2.) XXX Triglyceride

The fat and/or oil component contains at least one type of XXX triglyceride having a Cₓ fatty acid residue X at positions 1 to 3 of glycerin. The XXX triglyceride is a triglyceride having a fatty acid residue X with x carbon atoms at positions 1 to 3 of glycerin, and the three fatty acid residues are all identical to each other. Here, the number of carbon atoms x is an integer of 10 to 22, more preferably an integer of 12 to 22, even more preferably an integer of 14 to 20, and particularly preferably an integer of 16 to 18.

The fatty acid residue X may be a saturated or unsaturated fatty acid residue. Specific examples of the fatty acid residue X include, but are not limited to, capric acid, lauric acid, myristic acid, palmitic acid, stearic acid, arachidic acid, behenic acid, and like residues. The fatty acids are preferably lauric acid, myristic acid, palmitic acid, stearic acid, arachidic acid, and behenic acid; more preferably myristic acid, palmitic acid, stearic acid, and arachidic acid; and even more preferably palmitic acid, and stearic acid.

The lower limit of the XXX triglyceride content is, for example, 50 mass %, preferably 60 mass %, more preferably 70 mass %, even more preferably 80 mass %, and the upper limit of the XXX triglyceride content is, for example, 100 mass %, preferably 99 mass %, and more preferably 95 mass %, based on the total mass of the fat and/or oil component of the fat and/or oil particles defined as 100 mass %. One or two or more types of XXX triglycerides can be used. Preferably one or two types of XXX triglycerides are used, and more preferably one type of XXX triglyceride is used. When two or more types of XXX triglycerides are used, the XXX triglyceride content refers to the sum of the contents of the two or more types of XXX triglycerides.

### 1.2.3) Other Triglycerides

The fat and/or oil particle may contain a triglyceride other than the above-described XXX triglyceride as an oily component. Such other triglycerides can be two or more triglycerides other than the above-described triglyceride, and can be synthetic fats and/or oils or natural fats and/or oils. Examples of synthetic fats and/or oils include glyceryl tricaprylate, glyceryl tricaprate, and the like. Examples of natural fats and/or oils include cocoa butter, sunflower seed oil, rapeseed oil, soybean oil, cottonseed oil, and the like. A content of other triglycerides of 1 mass % or more, for example, about 5 to 50 mass %, based on the total amount of the triglycerides in the fat and/or oil particles defined as 100 mass %, does not cause any problems. The content of other triglycerides is, for example, 0 to 30 mass %, preferably 0 to 18 mass %, more preferably 0 to 15 mass %, and even more preferably 0 to 8 mass %.

1.2.4) Other Components in Fat and/or Oil Particles

The fat and/or oil particles may optionally contain, in addition to the fat and/or oil component containing the triglyceride, other components (additives), such as an emulsifier, a flavor, a skim milk powder, a whole milk powder, a cocoa powder, sugar, dextrin, a sweetener, and a coloring agent. These optional components can also be externally added to the fat and/or oil particles. However, incorporating the optional components into the fat and/or oil particles beforehand enables these optional components to reliably and easily adhere onto a food base material. These other components can be used in any amount, as long as the effects of the present invention are not impaired. The content of such other components is preferably, for example, 0.001 to 70 mass %, more preferably 0.01 to 65 mass %, and even more preferably 0.1 to 30 mass %, based on the total mass of the fat and/or oil particles defined as 100 mass %.

The fat and/or oil particles in the fat and/or oil composition containing 3-hydroxybutyric acid of the present invention preferably substantially consist of the fat and/or oil component, and the fat and/or oil component preferably substantially consists of triglyceride. The term “substantially” means that when the total amount of the fat and/or oil particles is defined as 100 mass %, the amount of triglyceride contained in the components other than the fat and/or oil component or triglyceride contained in the fat and/or oil component of the fat and/or oil particles is, for example, 85 to 100 mass %, preferably 90 to 100 mass %, and more preferably 95 to 100 mass %.

The 3HB-containing composition contains 3HB and/or a salt of 3HB and the fat and/or oil particles described above. The content of the fat and/or oil particles is preferably 0.5 to 100 parts by mass, more preferably 1 to 90 parts by mass, even more preferably 3 to 50 parts by mass, and particularly preferably 5 to 20 parts by mass, based on 100 parts by mass of the total amount of the 3HB and/or the salt of 3HB.

Since the fat and/or oil particles are present in an amount of 0.5 parts by mass per 100 parts by mass of the total amount of the 3HB and/or the salt of 3HB, deliquescence of the 3HB and/or the salt of 3HB can be reduced. On the other hand, since the fat and/or oil particles are present in an amount of 100 parts by mass or less per 100 parts by mass of the total amount of the 3HB and/or the salt of 3HB, the sour taste of the 3HB-containing composition can be sufficiently reduced.

When 3HB crystals alone, rather than 3HB salts, are used in the 3HB-containing composition of the present invention, the content of the fat and/oil particles is preferably 1 to 90 parts by mass, preferably 3 to 50 parts by mass, and more preferably 5 to 20 parts by mass, per 100 parts by mass of 3HB.

On the other hand, when a salt of 3HB alone is used in the 3HB-containing composition of the present invention, the content of the fat and/or oil particles is preferably 1 to 90 parts by mass, preferably 3 to 50 parts by mass, and more preferably 5 to 20 parts by mass, per 100 parts by mass of 3HB.

Since 3HB and/or a salt of 3HB and fat and/or oil particles are incorporated in the proportions described above, the resulting composition is configured such that the 3HB and/or the salt of 3HB is surface-coated with the fat and/or oil particles. In this mode, the effects of the present invention can be more efficiently achieved.

### 1-3) Other Components

The 3HB-containing composition of the present invention also preferably contains other components as appropriate as long as the object and effects of the present invention are not impaired. Examples of such other components include, but are not limited to, silicon dioxide, calcium silicate, calcium stearate, cellulose, and derivatives of cellulose.

### 2) Method for Producing 3HB-Containing Composition

The method for producing the fat and/or oil particles is not particularly limited. Specifically, the fat and/or oil particles can be obtained by the following production method. The fat and/or oil particles can be obtained by: melting a raw material for fat and/or oil particles comprising at least one type of XXX triglyceride having a Cₓ fatty acid residue X at positions 1 to 3 of glycerin; and maintaining the melted raw material at a specific cooling temperature to cool and solidify the material, whereby fat and/or oil particles in the form of a powder can be obtained without using any special processing means, such as spraying or mechanical pulverization using a pulverizer such as a mill. More specifically, the fat and/or oil particles can be obtained, for example, by a method comprising the steps of:

(a) preparing a raw material for fat and/or oil particles comprising the XXX triglyceride;
optionally, (b) heating the raw material for fat and/or oil particles obtained in step (a) to melt the triglyceride contained in the raw material for fat and/or oil particles, thereby obtaining a melted raw material for fat and/or oil particles; and further
(d) cooling and solidifying the raw material for fat and/or oil particles to obtain fat and/or oil particles comprising β-form fat and/or oil and having a plate-like particle shape.
The fat and/or oil particles can also be produced by subjecting the solid obtained after cooling to a known pulverization means, for example, using a hammer mill or a cutter mill.

The cooling in step (d) is performed, for example, by cooling a melted raw material for fat and/or oil particles at a temperature that is lower than the melting point of β-form fat and/or oil of the fat and/or oil component contained in the raw material for fat and/or oil particles, and that is equal to or higher than the cooling temperature calculated from the following formula:

Cooling temperature(° C.)=number of carbon atoms×x6.6−68.

When the cooling is performed within this temperature range, β-form fat and/or oil can be efficiently produced and fine crystals can be formed, whereby fat and/or oil particles can be easily obtained. The term “fine” means that the primary particle (the smallest crystal) has a size of, for example, 20 μm or less, preferably 15 μm or less, and more preferably 10 μm. When the cooling is not performed within the above temperature range, there are cases in which beta-form fat and/or oil is not formed and a solid having voids with a more increased volume than fat and/or oil particles cannot be produced. Further, by cooling within the above temperature range, β-form fat and/or oil can be formed in a static state, and fat and/or oil particles having a plate-like shape can be produced. The average particle size of the fat and/or oil particles in the 3HB-containing composition of the present invention is the same as described above.

The fat and/or oil particles can be produced, for example, by the following steps:

(a) preparing a raw material for fat and/or oil particles containing at least one type of XXX triglyceride;

optionally, (b) heating or otherwise treating the raw material for fat and/or oil particles obtained in step (a) to dissolve the triglyceride(s) contained in the raw material for fat and/or oil particles to obtain a melted raw material for fat and/or oil particles; and

(d) cooling and solidifying the melted raw material for fat and/or oil particles to obtain fat and/or oil particles containing β-form fat and/or oil and having a plate-like particle shape.

Additionally, the method may comprise an optional step for promoting the powder formation, such as a seeding step (c1), a tempering step (c2), or a pre-cooling step (c3), as a step (c) between the above steps (b) and (d). Further in the above step (d), an impact (e.g., crushing, loosing, vibration, or sieving) can be added to a solid with voids obtained after cooling to obtain fat and/or oil particles.

The above steps (a) to (d) are explained below.

### (a) Step of Preparing Raw Material

The raw material for fat and/or oil particles containing XXX triglyceride to be prepared in step (a) can be produced based on a method for producing usual fats and oils, such as XXX triglycerides, which include at least one type of XXX triglyceride having a Cₓ fatty acid residue X at positions 1 to 3 of glycerin; or can be easily obtained from the market. Here, the XXX triglyceride specified by the number of carbon atoms x and the type of fatty acid residue X are identical to those of the ultimately obtained target fat and/or oil component except in terms of polymorphs. The raw material may contain β-form fat and/or oil. The content of β-form fat and/or oil may be, for example, 0.1 mass % or less, 0.05 mass % or less, or 0.01 mass % or less. However, when the raw material is heated or otherwise treated and thus becomes in a melted state, the β-form fat and/or oil is lost from the raw material. Therefore, the raw material to be used may be a raw material in a melted state. For example, when the raw material is in a melted state, the meaning of the raw material substantially containing no β-form fat and/or oil includes the case in which not only XXX triglycerides but also substantially the entire fat and/or oil component contains no B-form fat and/or oil. The presence of β-form fat and/or oil can be confirmed, for example, by observing diffraction peaks derived from β-form fat and/or oil by X-ray diffraction measurement or detecting β-form fat and/or oil by differential scanning calorimetry, as described above. In the case of “substantially containing no β-form fat and/or oil,” the amount of β-form fat and/or oil present can be speculated from the intensity proportions of the characteristic peak of β-form fat and/or oil and the characteristic peak of α-form fat and/or oil among X-ray diffraction peaks, i.e., [characteristic peak intensity of β-form fat and/or oil/(characteristic peak intensity of α-form fat and/or oil+characteristic peak intensity of β-form fat and/or oil)] (peak intensity ratio). The peak intensity ratio of the raw material for fat and/or oil particles is, for example, preferably 0.2 or less, preferably 0.15 or less, and more preferably 0.10 or less. The raw material for fat and/or oil particles can contain one type or two or more types of XXX triglycerides described above, and preferably contains one type or two types of XXX triglycerides, and more preferably contains one type of XXX triglyceride.

Specifically, for example, the XXX triglyceride can be produced by direct synthesis using a fatty acid or a fatty acid derivative and glycerin. The method for directly synthesizing the XXX triglyceride includes:

(i) a method of directly esterifying a Cₓ fatty acid and glycerin (direct esterification synthesis);
(ii) a method of reacting glycerin with a fatty acid alkyl ester wherein a carboxyl group of a Cₓ fatty acid X is bonded to an alkoxyl group (e.g., fatty acid methyl and fatty acid ethyl) under basic or acidic catalyst conditions (transesterification synthesis using fatty acid alkyl); and
(iii) a method of reacting glycerin with a fatty acid halide wherein a hydroxyl group of a carboxyl group of the Cₓ fatty acid X is substituted with a halogen (e.g., fatty acid chloride and fatty acid bromide) in the presence of a basic catalyst (acid halide synthesis).

The XXX triglyceride can be produced by any of the above methods (i) to (iii). From the viewpoint of ease of production, (i) the direct esterification synthesis or (ii) the transesterification synthesis using a fatty acid alkyl ester is preferable, and the direct esterification synthesis (i) is more preferably used to produce the XXX triglyceride.

In order to produce the XXX triglyceride by the direct esterification synthesis (i), the fatty acid X or fatty acid Y is preferably used in an amount of 3 to 5 moles, and more preferably 3 to 4 moles, per mole of glycerin, from the viewpoint of production efficiency.

The reaction temperature of the XXX triglyceride in the direct esterification synthesis (i) can be any temperature at which water generated by the esterification reaction can be removed to the outside of the system. For example, the temperature is preferably 120° C. to 300° C., more preferably 150° C. to 270° C., and even more preferably 180° C. to 250° C. When the reaction is performed at 180 to 250° C., the XXX triglyceride can be particularly efficiently produced.

In the direct esterification synthesis (i) of the XXX triglyceride, a catalyst that promotes the esterification reaction can be used. Examples of usable catalysts include acidic catalysts, alkaline earth metal alkoxides, and the like. The amount of catalyst used is preferably about 0.001 to 1 mass % based on the total mass of the reaction raw materials.

In the direct esterification synthesis (i) of the XXX-triglyceride, the catalyst and unreacted raw materials can be removed, after the reaction, by performing a known purification treatment, such as washing with water, alkaline deacidification and/or deacidification under reduced pressure, and adsorption treatment. Further, decolorization and deodorization treatments can be performed to further purify the obtained product.

When the total mass of the triglycerides contained in the raw material for fat and/or oil particles is defined as 100 mass %, the amount of the XXX triglyceride is preferably 50 to 100 mass %, more preferably 55 to 95 mass %, even more preferably 60 to 90 mass %, and particularly preferably 65 to 85 mass %.

Examples of the raw material for fat and/or oil component contained in the fat and/or oil particles containing XXX triglyceride include commercially available triglyceride compositions and commercially available synthetic fats and/or oils. Examples of triglyceride compositions include hard palm stearin (produced by The Nisshin OilliO Group, Ltd.), highly hydrogenated rapeseed oil (produced by Yokozeki Oil & Fat Industries Co., Ltd.), and highly hydrogenated soybean oil (produced by Yokozeki Oil & Fat Industries Co., Ltd.). Examples of synthetic fats and/or oils include tripalmitin (produced by Tokyo Chemical Industry Co., Ltd.), tristearin (produced by Sigma-Aldrich Corporation), tristearin (produced by Tokyo Chemical Industry Co., Ltd.), triarachidin (produced by Tokyo Chemical Industry Co., Ltd.), and tribehenin (produced by Tokyo Chemical Industry Co., Ltd.). In addition, highly hydrogenated palm oil, which has a low XXX triglyceride content, can be used as a component for diluting the triglyceride.

The fat and/or oil particle containing XXX triglyceride may optionally contain other components, such as partial glycerides, fatty acids, antioxidants, emulsifiers, and solvents such as water, as described above.

When the raw material for fat and/or oil particles containing XXX triglyceride contains multiple components, the components can be optionally mixed. Any known mixing method can be used for the mixing operation, as long as a homogeneous reaction substrate is obtained. The mixing operation can be performed, for example, using a paddle mixer, an agi-homo mixer, or a disper mixer.

The mixing operation may be performed under heating as necessary. The heating is preferably performed at a heating temperature similar to that in step (b) to be described below. For example, the heating is preferably performed at 50 to 120° C., more preferably 60 to 100° C., even more preferably 70 to 90° C., particularly preferably 78 to 82° C., and most preferably 80° C.

(b) Step of Obtaining the Material for Fat and/or Oil Particles in a Melted State

Before the above step (d), if the raw material for fat and/or oil particles containing XXX triglyceride prepared in step (a) is in a melted state when prepared, the raw material for fat and/or oil particles is preferably directly cooled without heating. If the raw material for fat and/or oil particles is not in a melted state when prepared, it is preferable that the raw material for fat and/or oil particles is optionally heated to melt the triglyceride contained in the raw material for fat and/or oil particles, thus obtaining a raw material for fat and/or oil particles in a melted state.

Here, the raw material for fat and/or oil particles is preferably heated to a temperature equal to or higher than the melting point of triglyceride contained in the raw material for fat and/or oil particles, and more preferably heated to a temperature at which XXX triglyceride can be melted. The heating temperature is preferably 70 to 200° C., more preferably 75 to 150° C., and even more preferably 80 to 100° C. The heating can be continued, for example, for 0.1 to 3 hours, preferably 0.3 to 2 hours, and more preferably 0.5 to 1 hour.

(d) Step of Cooling the Raw Material for Fat and/or Oil Particles in a Melted State to Obtain Fat and/or Oil Particles

The raw material for fat and/or oil particles containing XXX triglyceride in a melted state prepared in step (a) or (b) above is preferably further cooled and solidified to form fat and/or oil particles containing β-form fat and/or oil and having a plate-like particle shape.

In order to “cool and solidify the raw material for fat and/or oil particles in a melted state,” the upper limit of the cooling temperature for cooling the raw material for fat and/or oil particles in a melted state is preferably kept lower than the melting point of β-form fat and/or oil of the fat and/or oil component contained in the raw material for fat and/or oil particles. For example, consider the case where the XXX triglyceride has three stearic acid residues each having 18 carbon atoms. In this case, since the melting point of β-form fat and/or oil is 74° C. (Table 1), the “temperature lower than the melting point of the β-form fat and/or oil of the fat and/or oil component contained in the raw material for fat and/or oil particles” is preferably a temperature lower by 1 to 30° C. than the melting point (i.e., 44 to 73° C.), more preferably a temperature lower by 1 to 20° C. than the melting point (i.e., 54 to 73° C.), even more preferably a temperature lower by 1 to 15° C. than the melting point (i.e., 59 to 73° C.), and particularly preferably a temperature lower by 1 to 10° C. than the melting point (i.e., 64 to 73° C.).

In order to obtain β-form fat and/or oil, the lower limit of the cooling temperature is preferably kept equal to or higher than the cooling temperature obtained from the following formula:

Cooling temperature(° C.)=Number of carbon atoms×x6.6-68

(wherein the number of carbon atoms x represents the number of carbon atoms x of the XXX triglyceride contained in the raw material for fat and/or oil particles).

The cooling temperature is set to a temperature equal to or higher than the temperature calculated above because the temperature must be set to a temperature at which α-form fat and/or oil and β′-form fat and/or oil, i.e., fats and/or oils other than β-form fat and/or oil, do not crystalize when the B-form fat and/or oil is crystalized to obtain β-form fat and/or oil containing XXX triglyceride. Since the cooling temperature mainly depends on the size of the molecule of XXX triglyceride, it can be understood that there is a certain correlation between the number of carbon atoms x and the lower limit of the optimum cooling temperature.

For example, when the XXX triglyceride contained in the raw material for fat and/or oil particles is a XXX triglyceride having three stearic acid residues each having 18 carbon atoms, the lower limit of the cooling temperature is 50.8° C. or more. Accordingly, when the XXX triglyceride has three stearic acid residues each having 18 carbon atoms, the temperature of “cooling and solidifying the raw material for fat and/or oil particles in a melted state” is more preferably 50.8° C. or more and 72° C. or less.

When the XXX triglyceride is a mixture of two or more types of XXX triglycerides, the lower limit of the cooling temperature of the mixture is preferably set in accordance with the cooling temperature of the XXX triglyceride having the smallest number of carbon atoms x. For example, when the XXX triglyceride contained in the raw material for fat and/or oil particles is a mixture of a XXX triglyceride having three palmitic acid residues each having 16 carbon atoms and a XXX triglyceride having three stearic acid residues each having 18 carbon atoms, the lower limit of the cooling temperature is preferably set to a temperature of 37.6° C. or higher in accordance with the cooling temperature of the XXX triglyceride having 16 carbon atoms, which is the smaller number of carbon atoms.

In another embodiment, it is appropriate to set the lower limit of the cooling temperature to a temperature equal to or higher than the melting point of the α-form fat and/or oil corresponding to the β-form fat and/or oil of the raw material for fat and/or oil particles containing XXX triglyceride. For example, when the XXX triglyceride contained in the raw material for fat and/or oil particles is a XXX triglyceride having three stearic acid residues each having 18 carbon atoms, the temperature of “cooling and solidifying the raw material for fat and/or oil particles in a melted state” is preferably 55° C. or more and 72° C. or less, because the melting point of the α-form fat and/or oil containing the XXX triglyceride having three stearic acid residues is 55° C. (Table 1).

In still another embodiment, for example, when x is 10 to 12, cooling of the raw material for fat and/or oil particles containing XXX triglyceride in a melted state is preferably performed to achieve a final temperature of −2 to 46° C., more preferably 12 to 44° C., and even more preferably 14 to 42° C. For example, when x is 13 or 14, the final temperature in the cooling is preferably 24 to 56° C., more preferably 32 to 54° C., and even preferably 40 to 52° C. When x is 15 or 16, the final temperature in the cooling is preferably 36 to 66° C., more preferably 44 to 64° C., and even more preferably 52 to 62° C. When x is 17 or 18, the final temperature in the cooling is preferably 50 to 72° C., more preferably 54 to 70° C., and even more preferably 58 to 68° C. When x is 19 or 20, the final temperature in the cooling is preferably 62 to 80° C., more preferably 66 to 78° C., and even more preferably 70 to 77° C. When x is 21 or 22, the final temperature in the cooling can be preferably 66 to 84° C., more preferably 70 to 82° C., and even more preferably 74 to 80° C. The raw material for fat and/or oil particles is preferably allowed to stand at the final temperature, for example, for 2 hours or more, more preferably 4 hours or more, and even more preferably 6 hours or more; and preferably 2 days or less, more preferably 24 hours or less, and even more preferably 12 hours or less.

### (c) Step of Promoting Powder Formation

The method preferably further comprises an optional step of (c) performing a powder formation-promoting step before step (d), between step (a) or (b) and step (d). In step (c), the raw material for fat and/or oil particles in a melted state, which is to be used in step (d), may be subjected to a treatment by seeding, tempering, or pre-cooling. That is, the step (c) is preferably (c1) a seeding process, (c2) a tempering process, or (c3) a pre-cooling process. These steps (c1) to (c3) can be performed singly or in a combination of two or more. The phrase “between step (a) or (b) and step (d)” as used herein is meant to include all of the following: “during step (a) or (b),” “after step (a) or (b) but before step (d),” and “during step (d).”

The seeding process and the tempering process are powder formation-promoting methods comprising treating the raw material for fat and/or oil particles in a melted state in order to securely form the melted raw material for fat and/or oil particles into a powder before cooling to the final temperature in the production of fat and/or oil particles contained in the 3HB-containing fat and/or oil composition of the present invention. Here, the seeding process is a method for promoting powdering by adding a small amount of a component that serves as a core (seed) of powder at the time of cooling the raw material for fat and/or oil particles in a melted state. Specifically, the seeding process is a method for promoting powdering of the raw material for fat and/or oil particles, for example, by the following steps. A fat and/or oil powder that contains preferably at least 80 mass %, more preferably at least 90 mass %, of XXX triglyceride having the same number of carbon atoms as the XXX triglyceride contained in the raw material for fat and/or oil particles is prepared as a component that serves as the core (seed) in the melted raw material for fat and/or oil particles obtained in step (b). When the temperature of the raw material for fat and/or oil particles has reached, for example, a temperature in the range of the final cooling temperature±0 to +10° C., preferably a temperature in the range of the final cooling temperature+5 to +10° C., during the cooling of the raw material for fat and/or oil particles in a melted state, the fat and/or oil powder that serves as the core is added in an amount of 0.1 to 1 part by mass, preferably 0.2 to 0.8 parts by mass, per 100 parts by mass of the melted raw material for fat and/or oil particles.

The tempering process is a method wherein in the cooling of the melted raw material for fat and/or oil particles, before being allowed to stand at a final cooling temperature, the raw material is cooled once at a temperature lower than the cooling temperature used in step (d), for example, at a temperature lower by 5 to 20° C., preferably a temperature lower by 7 to 15° C., and more preferably a temperature lower by about 10° C., than the cooling temperature used in step (d), preferably for 10 to 120 minutes, and more preferably for about 30 to 90 minutes, to thereby promote powdering of the raw material for fat and/or oil particles.

The pre-cooling process is a method wherein before the cooling in step (d), the melted raw material for fat and/or oil particles obtained in step (a) or (b) is once pre-cooled at a temperature between the temperature used at the time of preparing the raw material for fat and/or oil particles containing XXX triglyceride and the cooling temperature used at the time of cooling the raw material for fat and/or oil particles. In other words, the pre-cooling process is a method for pre-cooling once at a temperature that is lower than the temperature of achieving the melted state in step (a) or (b) and that is higher than the cooling temperature in step (d). After the pre-cooling step (c3), cooling is preferably performed at the cooling temperature used at the time of cooling the raw material for fat and/or oil particles in step (d). The temperature higher than the cooling temperature of step (d) can be, for example, a temperature higher by 2 to 40° C., preferably a temperature higher by 3 to 30° C., more preferably a temperature higher by 4 to 30° C., and even more preferably a temperature higher by about 5 to 10° C., than the cooling temperature in step (b). As the pre-cooling temperature is set to a lower temperature, the main cooling time at the cooling temperature in step (d) can be set to a shorter time.

Thus, unlike the seeding process or the tempering process, the pre-cooling process is a method capable of promoting powdering of the raw material for fat and/or oil particles by simply lowering the cooling temperature stepwise, and has a great advantage in industrial production.

The solid with voids obtained after cooling in step (d) is preferably a solidified product with voids having an increased volume as compared with that of the raw material for fat and/or oil particles in a melted state. Since the solid with voids easily collapses into a powder material, a powder material can be obtained, even without particularly providing a powdering step, by collapse of voids during a step of filling a container with the solid with voids or a transportation step.

To explain this in detail, preferably, the raw material for fat and/or oil particles containing XXX triglyceride is first melted to obtain a melted raw material for fat and/or oil particles, and then cooled to form a solid with voids having an increased volume as compared with that of the melted raw material for fat and/or oil particles. The raw material for fat and/or oil particles that is formed into a solid with voids can easily be pulverized by a mild impact and easily collapses into a powder form.

The method for giving an impact is not particularly limited. Examples include a method of pulverizing a solid with voids using a usual pulverizer (such as a hammer mill or a cutter mill); a method of loosening a solid with voids using a spatula, a rubber spatula, a shovel, or the like; a method of shaking a solid with voids in a container; and a method of sieving a solid with voids; and the like.

The compositions containing 3HB can be obtained, for example, by mixing 3HB and/or a salt thereof and fat and/or oil particles optionally with other components.

The mixing method is not limited, and a known method can be used for mixing. For example, mechanical mixing using a mixer or a mill can be used.

Examples of other methods for obtaining the 3HB-containing composition of the present invention include a method comprising mixing and pulverizing the fat and/or oil particles and 3HB crystals, and then evaporating (volatilizing) the water remaining in the mixture by using an evaporator or the like.

Embodiments of the present invention are described above. However, the present invention is not limited to such embodiments and can, of course, be carried out in various configurations without departing from the gist of the present invention.

### EXAMPLES

Embodiments of the present invention are described in more detail below based on Examples. However, the present invention is not limited thereto or thereby.

### Analysis Method

### Triglyceride Composition

Gas chromatographic analysis conditions
DB1-ht (0.32 mm×0.1 μm×5 m) produced by Agilent Technologies (123-1131)
Injection volume: 1.0 μL

### Inlet: 370° C.

### Detector: 370° C.

Split ratio: 50/1 35.1 kPa, constant pressure
Column CT: 200° C. (0 min hold) to (15° C./min) to 370° C. (4 min hold).

### X-Ray Diffraction Measurement

An X-ray diffraction measurement was performed with an Ultima IV X-ray diffractometer (produced by Rigaku Co., Ltd.) using CuKα (λ=1.542 Å) as a radiation source and using a Cu filter under the following conditions: a power output of 1.6 kW, an operating angle of 0.96 to 30.0°, and a measurement speed of 2°/min. In this measurement, when only the peak around 4.6 Å was present and no peaks were observed around 4.1 to 4.2 Å, it was determined that the fat and/or oil component consisted only of B-form fat and/or oil.

From the results of the X-ray diffraction measurement, the peak intensity ratio=[intensity of characteristic peak of β-form fat and/or oil (2θ=19° (4.6 Å))/(intensity of characteristic peak of α-form fat and/or oil (2θ=21° (4.2 Å))+intensity of characteristic peak of β-form fat and/or oil (2θ=19° (4.6 Å)))] was measured as an index for the amount of β-form fat and/or oil present.

### Loose Bulk Density

The loose bulk density of fat and/or oil particles (g/cm³) was determined by letting the fat and/or oil particles fall approximately 2 cm above the upper-opening end of a 25-mL graduated cylinder with an inner diameter 15 mm to loosely fill the graduated cylinder with the fat and/or oil particles; measuring the mass (g) of the fat and/or oil particles in the cylinder and reading the volume (mL) of the fat and/or oil particles in the cylinder; and calculating the mass (g) of the fat and/or oil particles per cm³.

### Aspect Ratio

By direct observation with an S-3400N scanning electron microscope (produced by Hitachi High-Technologies Corporation) and using software for measuring particle size distribution through image analysis (Mac-View, produced by Mountech Co., Ltd.), arbitrarily selected particles were measured for lengths in the major-axis and minor-axis directions, and the average of the measured particles was obtained.

### Average Particle Diameter

The average particle diameter of the obtained fat and/or oil particles was determined by wet measurement based on laser diffraction/scattering methods (IS0133201, ISO9276-1) using a particle size distribution analyzer (Microtrac MT3300 Ex II, produced by Nikkiso Co., Ltd.).

Specifically, a very small-volume circulator (equipment name: USVR, produced by Nikkiso Co., Ltd.) was attached to the particle size distribution analyzer, and water was circulated as a dispersion solvent. 0.06 g of a sample and 0.6 g of a neutral detergent were placed in a 100-ml beaker and mixed with a spatula. After the mixing, 30 ml of water was added and the resulting mixture was subjected to an ultrasonic cleaner (equipment name: AU-16C, produced by Aiwa Medical Industry Co., Ltd.) for 1 minute. The resulting mixture was added dropwise to the circulator and circulated for measurement. The measured particle size at a cumulative percentage of 50% in the obtained particle size distribution (d50) was used as the average particle size.

Preparation of Fat and/or Oil Particles

1 kg of a triglyceride having a stearic acid residue (having 18 carbon atoms) at positions 1 to 3 (XXX triglyceride: 79.1 mass %, highly hydrogenated rapeseed oil, produced by Yokozeki Oil & Fat Industries Co., Ltd.) was maintained at 80° C. for 10 hours and thereby completely melted. The melted triglyceride was cooled in a thermostatic chamber at 60° C. for 15 hours to form a solid with voids having an increased volume. After completion of crystallization, cooling was performed until a room temperature (25° C.) condition was achieved. The obtained solid was pulverized with a hammer mill to obtain a powder fat and/or oil (melting point: 67.4° C., loose bulk density: 0.2 g/cm³, aspect ratio 1.6, average particle diameter: 14.4 μm, X-ray diffraction measurement diffraction peak: 4.6 Å, peak intensity ratio: 0.89). This was used as fat and/or oil particles.

### Examples 1 to 21 and Comparative Examples 1 to 6

R-3HB crystals or R-3HB salts (3HB-Na: sodium salt, 3HB-K: potassium salt) and oil and fat particles were weighed in the proportions shown in Table 2 below. Each mixture thereof was milled for 3 seconds five times in a mill mixer (IJM-M800-W, produced by Iris Ohyama Inc.). 1 g of each of the 3HB-containing compositions obtained in the Examples and Comparative Examples was weighed in petri dishes and left uncovered in a room adjusted to 25° C. for 1 week. The appearance and weight change of each composition was then evaluated. As comparative examples, combinations of commonly used silicon dioxide or calcium silicate and fat and/or oil were evaluated. Table 2 below shows the results. The evaluation was made according to the following criteria.

o: High fluidity and silky smoothness.
Δ: Slightly moist but highly fluid powder.
x: Agglomeration or deliquescence occurred.

TABLE 2
Table 2

| | | Silicon | | | | | | | | Weight |
|---|---|---|---|---|---|---|---|---|---|---|
| | Fat and/ | dioxide | | | Potassium | Sodium | Appearance | Appearance | Appearance | change |
| | or oil | micropar- | Calcium | 3HB | salt of | salt of | immediately | after | after | after |
| | particles/g | ticles/g | silicate/g | crystals/g | 3HB/g | 3HB/g | after milling | 1 day | 1 week | 1 week |
| Example 1 | 5 | | | 5 | | | ∘ | ∘ | ∘ | 0.00 |
| Example 2 | 2.5 | | | 5 | | | ∘ | ∘ | ∘ | 0.01 |
| Example 3 | 1 | | | 5 | | | ∘ | ∘ | ∘ | 0.00 |
| Example 4 | 0.25 | | | 5 | | | ∘ | ∘ | ∘ | 0.00 |
| Example 5 | 0.1 | | | 5 | | | ∘~Δ | ∘~Δ | ∘~Δ | −0.02 |
| Example 6 | 5 | | | | 5 | | ∘ | ∘ | ∘ | 0.19 |
| Example 7 | 4 | | | | 5 | | ∘ | ∘ | ∘ | 0.23 |
| Example 8 | 3 | | | | 5 | | ∘ | ∘ | ∘ | 0.24 |
| Example 9 | 2 | | | | 5 | | ∘ | ∘~Δ | x | 0.25 |
| Example 10 | 5 | | | | | 5 | ∘ | ∘ | ∘ | 0.04 |
| Example 11 | 2 | | | | | 5 | ∘ | ∘ | ∘ | 0.05 |
| Example 12 | 1 | | | | | 5 | ∘ | ∘ | ∘ | 0.05 |
| Example 13 | 0.5 | | | | | 5 | ∘ | ∘ | ∘~Δ | 0.05 |
| Example 14 | 5 | | | 4 | 1 | | ∘ | ∘ | ∘ | 0.02 |
| Example 15 | 5 | | | 3 | 2 | | ∘ | ∘ | ∘ | 0.05 |
| Example 16 | 5 | | | 2 | 3 | | ∘ | ∘ | ∘ | 0.08 |
| Example 17 | 5 | | | 1 | 4 | | ∘ | ∘ | ∘ | 0.12 |
| Example 18 | 5 | | | 4 | | 1 | ∘ | ∘ | ∘ | 0.04 |
| Example 19 | 5 | | | 3 | | 2 | ∘ | ∘ | ∘ | 0.04 |
| Example 20 | 5 | | | 2 | | 3 | ∘ | ∘ | ∘ | 0.04 |
| Example 21 | 5 | | | 1 | | 4 | ∘ | ∘ | ∘ | 0.04 |
| Comp. Ex. 1 | | | | 5 | | | Δ | x (Partial | x (Partial | — |
| | | | | | | | | deliquescence) | deliquescence) | |
| Comp. Ex. 2 | | | | | 5 | | Δ | x | x | — |
| | | | | | | | | (Deliquescence) | (Deliquescence) | |
| Comp. Ex. 3 | | | | | | 5 | ∘ | x | x | — |
| | | | | | | | | (Deliquescence) | (Deliquescence) | |
| Comp. Ex. 4 | | 0.25 | | 5 | | | ∘ | x | x | — |
| | | | | | | | | (Agglomeration) | (Agglomeration) | |
| Comp. Ex. 5 | | | 0.25 | 5 | | | ∘ | x | x | — |
| | | | | | | | | (Agglomeration) | (Agglomeration) | |
| Comp. Ex. 6 | | | 0.25 | | 5 | | ∘ | x | x | — |
| | | | | | | | | (Deliquescence) | (Deliquescence) | |

As a result of the test, a powder with reduced deliquescence even one week after the preparation, which was easy to handle, was obtained by mechanically mixing R-3HB and/or a potassium salt or a sodium salt of R-3HB and fat and/or oil particles. When silicon dioxide and calcium silicate commonly used for anti-caking purpose, which were prepared for comparison, were added to R-3HB in an amount of 5%, partial agglomeration or deliquescence was observed after 1 week. These anti-caking agents were unsuitable as anti-caking agents for R-3HB and/or the potassium salt of R-3HB because such anti-caking agents are usually only allowed to be added to up to 2% as food additives. Compositions containing silicon dioxide microparticles or calcium silicate were difficult to handle because immediately after the preparation, static electricity was generated from the compositions and the powder scattered. Three expert panelists evaluated the sour taste of the compositions obtained in the Examples and Comparative Examples. All three panelists evaluated all of the Examples as having a reduced sour taste, while all of the Comparative Examples were evaluated as having a sour taste.

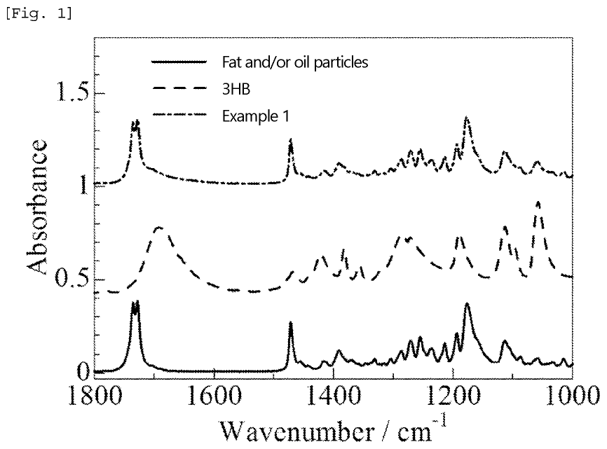

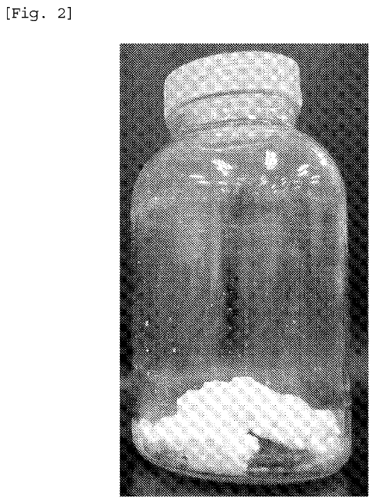

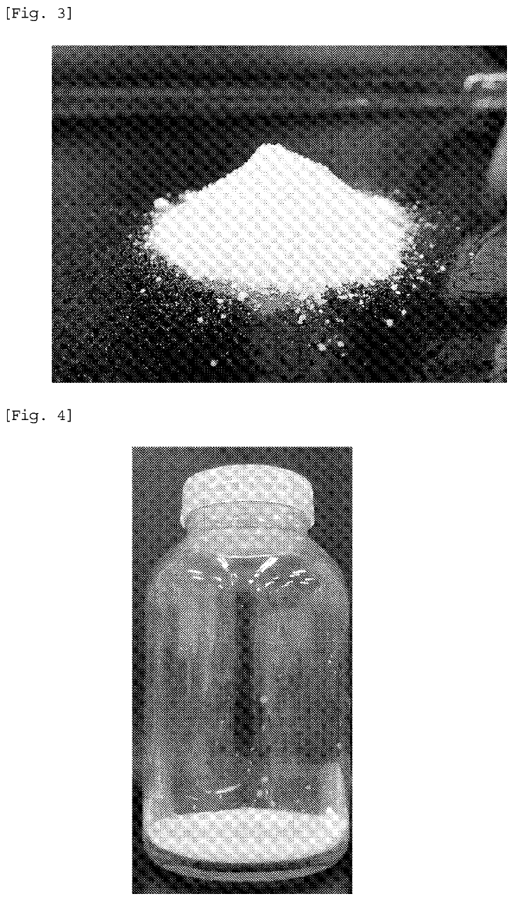

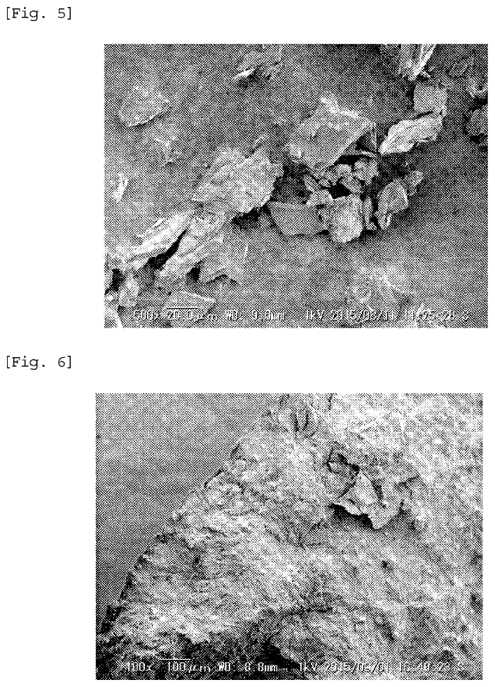

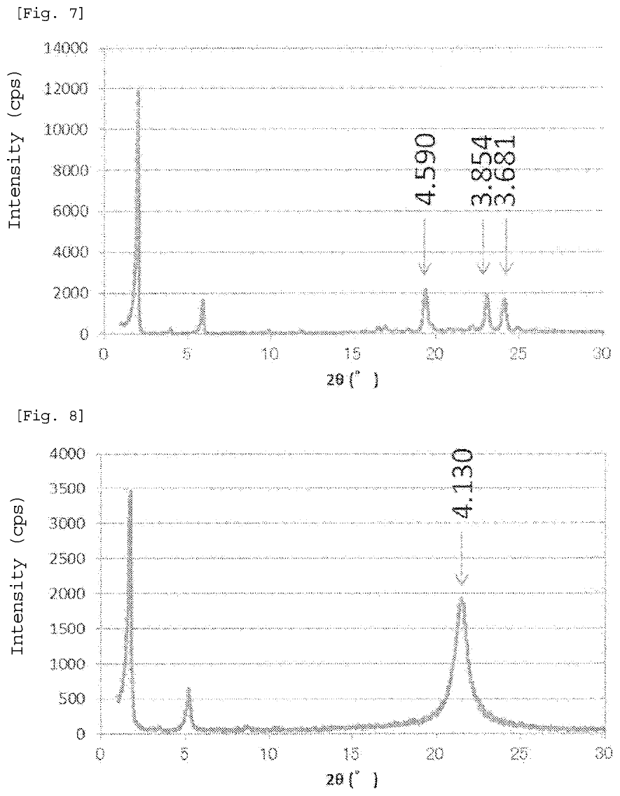

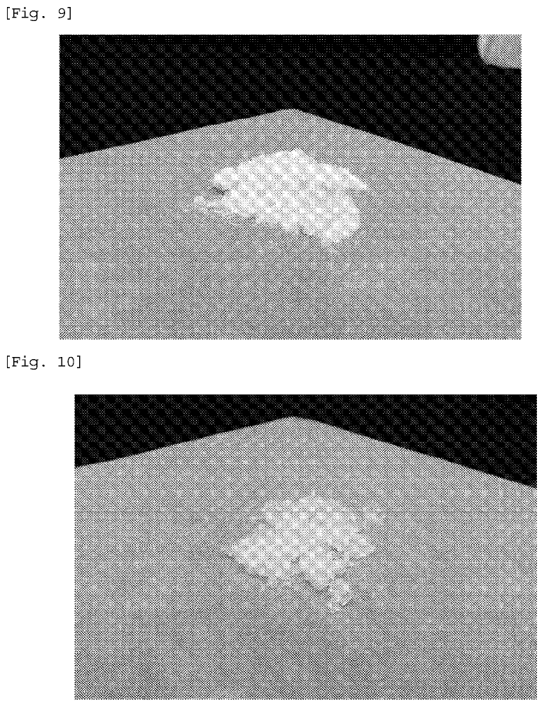

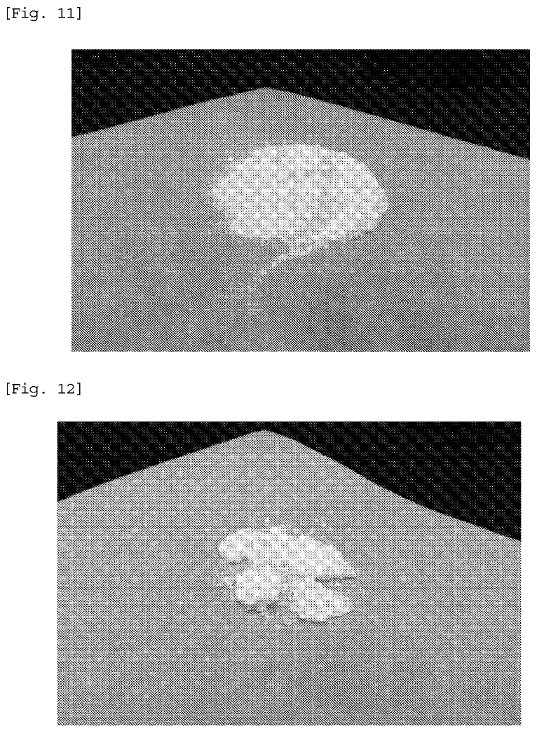

FIG. 1 shows the results of FT-IR measurements (iS5, produced by Thermo Fisher Scientific) of R-3HB crystals, fat and/or oil particles, and the composition obtained in Example 1 by the ATR method. The results revealed that the spectrum of Example 1 is almost identical to that of the fat and/or oil particles, and R-3HB was thus found to be coated with the fat and/or oil particles. This is considered to be a factor in the suppression of deliquescence and sour taste of R-3HB and/or salts of R-3HB.

Further, Production Examples for producing the fat and/or oil particles of the present invention are described below. The powder fat and/or oil particles obtained in the Production Examples below can also be used in the same manner as in the Examples described above.

### Production Example 1: x=16

25 g of a triglyceride having palmitic acid residues (each having 16 carbon atoms) at positions 1 to 3 (XXX triglyceride: 89.7 mass %, tripalmitin, produced by Tokyo Chemical Industry Co., Ltd.) was maintained at 80° C. for 0.5 hours and thereby completely melted. The melted triglyceride was cooled in a thermostatic chamber at 50° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 2.0, average particle diameter: 119 μm, X-ray diffraction measurement diffraction peak: 4.6 Å, peak intensity ratio: 0.90).

### Production Example 2: x=16

25 g of a triglyceride having palmitic acid residues (each having 16 carbon atoms) at positions 1 to 3 (XXX triglyceride: 69.9 mass %, hard palm stearin, produced by The Nisshin OilliO Group, Ltd.) was maintained at 80° C. for 0.5 hours and thereby completely melted. The melted triglyceride was cooled in a thermostatic chamber at 50° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.3 g/cm³, aspect ratio: 1.4, average particle diameter: 99 μm, X-ray diffraction measurement diffraction peak: 4.6 Å, peak intensity ratio: 0.88).

### Production Example 3: x=16, Tempering Process (c2)

15 g of a triglyceride having palmitic acid residues (each having 16 carbon atoms) at positions 1 to 3 (XXX triglyceride: 89.7 mass %, tripalmitin, produced by Tokyo Chemical Industry Co., Ltd.) was maintained at 80° C. for 0.5 hours and thereby completely melted. The melted triglyceride was cooled in a 30° C. thermostatic chamber for 0.01 hours and then allowed to stand in a 60° C. thermostatic chamber for 2 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 2.0, average particle diameter: 87 μm, X-ray diffraction measurement diffraction peak: 4.6 Å, peak intensity ratio: 0.89).

### Production Example 4: x=16, Seeding Process (c1)

First, 15 g of a triglyceride having palmitic acid residues (each having 16 carbon atoms) at positions 1 to 3 (XXX triglyceride: 89.7 mass %, tripalmitin, produced by Tokyo Chemical Industry Co., Ltd.) was maintained at 80° C. for 0.5 hours and thereby completely melted. The melted triglyceride was cooled in a thermostatic chamber at 60° C. until the product temperature had reached 60° C. Then, the tripalmitin fat and/or oil powder was added in an amount of 0.1 mass % to a raw material fat and/or oil. The resulting mixture was allowed to stand in the thermostatic chamber at 60° C. for 2 hours to form a solid with voids having an increased volume. After completion of crystallization, cooling was performed until a room temperature (25° C.) condition was achieved. The obtained solid was loosened to obtain a powdered crystalline composition (loose bulk density: 0.2 g/cm³, aspect ratio: 2.0, average particle diameter: 92 μm, X-ray diffraction measurement diffraction peak: 4.6 Å, peak intensity ratio: 0.89).

### Production Example 5: x=18

3 g of a triglyceride having stearic acid residues (each having 18 carbon atoms) at positions 1 to 3 (XXX triglyceride: 99.6 mass %, tristearin, produced by Sigma-Aldrich Corporation) was maintained at 80° C. for 0.5 hours and thereby completely melted. The melted triglyceride was cooled in a thermostatic chamber at 60° C. for 12 hours to obtain a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 2.0, average particle diameter: 30 μm, X-ray diffraction measurement diffraction peak: 4.6 Å, peak intensity ratio: 0.93).

### Production Example 6: x=18

25 g of a triglyceride having stearic acid residues (each having 18 carbon atoms) at positions 1 to 3 (XXX triglyceride: 96.0 mass %, tristearin, produced by Tokyo Chemical Industry Co., Ltd.) was maintained at 80° C. for 0.5 hours and thereby completely melted. The melted triglyceride was cooled in a thermostatic chamber at 55° C. for 12 hours to form a solid with voids having an increased volume. After completion of crystallization, cooling was performed until a room temperature (25° C.) condition was achieved. The obtained solid was loosened to obtain a powdered crystalline composition (loose bulk density: 0.2 g/cm³, aspect ratio: 2.0, average particle diameter: 31 μm, X-ray diffraction measurement diffraction peak: 4.6 Å, peak intensity ratio: 0.88).

### Production Example 7: x=18

25 g of a triglyceride having stearic acid residues (each having 18 carbon atoms) at positions 1 to 3 (XXX triglyceride: 79.1 mass %, highly hydrogenated rapeseed oil, produced by Yokozeki Oil & Fat Industries Co., Ltd.) was maintained at 80° C. for 0.5 hours and thereby completely melted. The melted triglyceride was cooled in a thermostatic chamber at 55° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 1.6, average particle diameter: 54 μm, X-ray diffraction measurement diffraction peak: 4.6 Å, peak intensity ratio: 0.89).

### Production Example 8: x=18

25 g of a triglyceride having stearic acid residues (each having 18 carbon atoms) at positions 1 to 3 (XXX triglyceride: 66.7 mass %, highly hydrogenated soybean oil, produced by Yokozeki Oil & Fat Industries Co., Ltd.) was maintained at 80° C. for 0.5 hours and thereby completely melted. The melted triglyceride was cooled in a thermostatic chamber at 55° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.3 g/cm³, aspect ratio: 1.4, average particle diameter: 60 μm, X-ray diffraction measurement diffraction peak: 4.6 Å, peak intensity ratio: 0.91).

### Production Example 9: x=18

A triglyceride having stearic acid residues (each having 18 carbon atoms) at positions 1 to 3 (XXX triglyceride: 84.1 mass %, Nisshin sunflower seed oil (S) (high oleic sunflower seed oil), produced by The Nisshin OilliO Group, Ltd.) was completely hydrogenated by a usual method to obtain a hydrogenated triglyceride (XXX triglyceride: 83.9 mass %). 25 g of the obtained highly hydrogenated oil of the high oleic sunflower seed oil was maintained at 80° C. for 0.5 hours and thereby completely melted. The melted triglyceride was cooled in a thermostatic chamber at 55° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 1.6, average particle diameter: 48 μm, X-ray diffraction measurement diffraction peak: 4.6 Å, peak intensity ratio: 0.89).

### Production Example 10: x=18

First, 18.75 g of a triglyceride having stearic acid residues (each having 18 carbon atoms) at positions 1 to 3 (XXX triglyceride: 66.7 mass %, highly hydrogenated soybean oil, produced by Yokozeki Oil & Fat Industries Co., Ltd.) was mixed with 6.25 g of another triglyceride having stearic acid residues (each having 18 carbon atoms) at positions 1 to 3 (XXX triglyceride: 11.1 mass %, highly hydrogenated palm oil, produced by Yokozeki Oil & Fat Industries Co. Ltd.) to obtain a raw material fat and/or oil (XXX triglyceride: 53.6 mass %). The raw material fat and/or oil was maintained at 80° C. for 0.5 hours and thereby completely melted. The melted triglyceride was cooled in a thermostatic chamber at 55° C. for 12 hours to thereby obtain a solid with voids having an increased volume. After completion of crystallization, cooling was performed until a room temperature (25° C.) condition was achieved. The obtained solid was loosened to obtain a crystalline powder composition (loose bulk density: 0.3 g/cm³, aspect ratio: 1.4, average particle diameter: 63 μm, X-ray diffraction measurement diffraction peak: 4.6 Å, peak intensity ratio: 0.78). The highly hydrogenated palm oil, which had a very low XXX triglyceride content, was used as a diluent component (the same applied hereinafter).

### Production Example 11: x=18, Seeding Process (c1)

25 g of a triglyceride having stearic acid residues (each having 18 carbon atoms) at positions 1 to 3 (XXX triglyceride: 96.0 mass %, tristearin, produced by Tokyo Chemical Industry Co., Ltd.) was maintained at 80° C. for 0.5 hours and thereby completely melted. The melted triglyceride was cooled in a thermostatic chamber at 70° C. until the product temperature had reached 70° C. A tristearin fat and/or oil powder was then added in an amount of 0.1 mass % to the raw material fat and/or oil, and the resulting mixture was allowed to stand in a thermostatic chamber at 70° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 2.0, average particle diameter: 36 μm, X-ray diffraction measurement diffraction peak: 4.6 Å, peak intensity ratio: 0.88).

### Production Example 12: x=18, Tempering Process (c2)

15 g of a triglyceride having stearic acid residues (each having 18 carbon atoms) at positions 1 to 3 (XXX triglyceride: 79.1 mass %, highly hydrogenated rapeseed oil, produced by Yokozeki Oil & Fat Industries Co., Ltd.) was maintained at 80° C. for 0.5 hours and thereby completely melted. The melted triglyceride was cooled in a thermostatic chamber at 50° C. for 0.1 hours and then allowed to stand in a thermostatic chamber at 65° C. for 6 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 1.6, average particle diameter: 50 μm, X-ray diffraction measurement diffraction peak: 4.6 Å, peak intensity ratio: 0.90).

### Production Example 13: x=18, Tempering Process (c2)

15 g of a triglyceride having stearic acid residues (each having 18 carbon atoms) at positions 1 to 3 (XXX triglyceride: 79.1 mass %, highly hydrogenated rapeseed oil, produced by Yokozeki Oil & Fat Industries Co., Ltd.) was maintained at 80° C. for 0.5 hours and thereby completely melted. The melted triglyceride was cooled in a thermostatic chamber at 40° C. for 0.01 hours and then allowed to stand in a thermostatic chamber at 65° C. for 2 hours to form a solid with voids having an increased volume. After completion of crystallization, cooling was performed until a room temperature (25° C.) condition was achieved. The obtained solid was loosened to obtain a powdered crystalline composition (loose bulk density: 0.2 g/cm³, aspect ratio: 1.6, average particle diameter: 52 μm, X-ray diffraction measurement diffraction peak: 4.6 Å, peak intensity ratio: 0.89).

### Production Example 14: x=18, Pre-Cooling Process (c3)

25 g of a triglyceride having stearic acid residues (each having 18 carbon atoms) at positions 1 to 3 (XXX triglyceride: 79.1 mass %, highly hydrogenated rapeseed oil, produced by Yokozeki Oil & Fat Industries Co., Ltd.) was maintained at 80° C. for 0.5 hours and thereby completely melted. The raw material fat and/or oil was maintained in a thermostatic chamber at 70° C. until the raw material fat and/or oil had reached 70° C. and then cooled in a thermostatic chamber at 65° C. for 8 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 1.6, average particle diameter: 60 μm, X-ray diffraction measurement diffraction peak: 4.6 Å, peak intensity ratio: 0.89).

### Production Example 15: x=20

10 g of a triglyceride having arachidic acid residues (each having 20 carbon atoms) at positions 1 to 3 (XXX triglyceride: 99.5 mass %, triarachidin, produced by Tokyo Chemical Industry Co., Ltd.) was maintained at 90° C. for 0.5 hours and thereby completely melted. The melted triglyceride was cooled in a thermostatic chamber at 72° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 2.0, average particle diameter: 42 μm, X-ray diffraction measurement diffraction peak: 4.6 Å, peak intensity ratio: 0.92).

### Production Example 16: x=22

10 g of a triglyceride having behenic acid residues (each having 22 carbon atoms) at positions 1 to 3 (XXX triglyceride: 97.4 mass %, tribehenin, produced by Tokyo Chemical Industry Co., Ltd.) was maintained at 90° C. for 0.5 hours and thereby completely melted. The melted triglyceride was cooled in a thermostatic chamber at 79° C. for 12 hours to form a solid with voids having an increased volume. After completion of crystallization, cooling was performed until a room temperature (25° C.) condition was achieved. The obtained solid was loosened to obtain a crystalline powder composition (loose bulk density: 0.2 g/cm³, aspect ratio: 2.0, average particle diameter: 52 μm, X-ray diffraction measurement diffraction peak: 4.6 Å, peak intensity ratio: 0.93).

### Production Example 17: x=16, 18

12.5 g of a triglyceride having palmitic acid residues (each having 16 carbon atoms) at positions 1 to 3 (XXX triglyceride: 89.7 mass %, tripalmitin, produced by Tokyo Chemical Industry Co., Ltd.) was mixed with 12.5 g of a triglyceride having stearic acid residues (each having 18 carbon atoms) at positions 1 to 3 (XXX triglyceride: 96.0 mass %, tristearin, produced by Tokyo Chemical Industry Co., Ltd.) to prepare a raw material fat and/or oil (XXX triglyceride: 93.8%). The raw material fat and/or oil was maintained at 80° C. for 0.5 hours and thereby completely melted. The melted raw material was cooled in a thermostatic chamber at 55° C. for 16 hours to obtain a solid with voids having an increased volume. The obtained solid was loosened to obtain a crystalline powder composition (loose bulk density: 0.2 g/cm³, aspect ratio: 1.6, average particle diameter: 74 μm, X-ray diffraction measurement diffraction peak: 4.6 Å, peak intensity ratio: 0.90).

### Production Example 18: x=16, 18

12.5 g of a triglyceride having palmitic acid residues (each having 16 carbon atoms) at positions 1 to 3 (XXX triglyceride: 69.9 mass %, hard palm stearin, produced by The Nisshin OilliO Group, Ltd.) was mixed with 12.5 g of a triglyceride having stearic acid residues (each having 18 carbon atoms) at positions 1 to 3 (XXX triglyceride: 79.1 mass %, highly hydrogenated rapeseed oil, produced by Yokozeki Oil & Fat Industries Co., Ltd.) to prepare a raw material fat and/or oil (XXX triglyceride: 75.3%). The raw fat and/or oil material was maintained at 80° C. for 0.5 hours and thereby completely melted. The melted raw material was cooled in a thermostatic chamber at 55° C. for 16 hours to form a solid with voids having an increased volume. The obtained solid was then loosened to obtain a crystalline powder composition (loose bulk density: 0.3 g/cm³, aspect ratio: 1.4, average particle diameter: 77 μm, X-ray diffraction measurement diffraction peak: 4.6 Å, peak intensity ratio: 0.88).

### Comparative Production Example 1: x=16

25 g of a triglyceride having palmitic acid residues (each having 16 carbon atoms) at positions 1 to 3 (XXX triglyceride: 89.7 mass %, tripalmitin, produced by Tokyo Chemical Industry Co., Ltd.) was maintained at 80° C. for 0.5 hours and thereby completely melted. The melted raw material was cooled in a thermostatic chamber at 25° C. for 4 hours and thereby completely solidified (X-ray diffraction measurement diffraction peak: 4.1 Å, peak intensity ratio: 0.10). No crystalline powder composition could be obtained.

### Comparative Production Example 2: x=16, 18

12.5 g of a triglyceride having palmitic acid residues (each having 16 carbon atoms) at positions 1 to 3 (XXX triglyceride: 69.9 mass %, hard palm stearin, produced by The Nisshin OilliO Group, Ltd.) was mixed with 12.5 g of a triglyceride having stearic acid residues (each having 18 carbon atoms) at positions 1 to 3 (XXX triglyceride: 11.1 mass %, highly hydrogenated palm oil, produced by Yokozeki Oil & Fat Industries Co., Ltd.) to prepare a raw material fat and/or oil (XXX triglyceride: 39.6%). The raw material fat and/or oil was maintained at 80° C. for 0.5 hours and thereby completely melted. The melted raw material was cooled in a thermostatic chamber at 40° C. for 12 hours and thereby completely solidified (X-ray diffraction measurement diffraction peak: 4.2 Å, peak intensity ratio: 0.12). No crystalline powder composition could be obtained.

### Comparative Production Example 3: x=18

25 g of a triglyceride having stearic acid residues (each having 18 carbon atoms) at positions 1 to 3 (XXX triglyceride: 79.1 mass %, highly hydrogenated rapeseed oil, produced by Yokozeki Oil & Fat Industries Co., Ltd.) was maintained at 80° C. for 0.5 hours and thereby completely melted. The melted raw material was cooled in a thermostatic chamber at 40° C. for 3 hours and thereby completely solidified (X-ray diffraction measurement diffraction peak: 4.1 Å, peak intensity ratio: 0.11). No crystalline powder composition could be obtained.

### Comparative Production Example 4: x=18

12.5 g of a triglyceride having stearic acid residues (each having 18 carbon atoms) at positions 1 to 3 (XXX triglyceride: 66.7 mass %, highly hydrogenated soybean oil, produced by Yokozeki Oil & Fat Industries Co., Ltd.) was mixed with 12.5 g of another triglyceride having stearic acid residues (each having 18 carbon atoms) at positions 1 to 3 (XXX triglyceride: 11.1 mass %, highly hydrogenated palm oil, produced by Yokozeki Oil & Fat Industries Co., Ltd.) to prepare a raw material fat and/or oil (XXX triglyceride: 39.7%). The raw fat and/or oil material was maintained at 80° C. for 0.5 hours and thereby completely melted. The melted raw material was cooled in a thermostatic chamber at 55° C. for 12 hours and thereby completely solidified (X-ray diffraction measurement diffraction peak: 4.2 Å, peak intensity ratio: 0.12). No crystalline powder composition could be obtained.

Table 3 summarizes the results of the above Production Examples and Comparative Production Examples.

TABLE 3

| | | | | | | | X-ray | | | |
|---|---|---|---|---|---|---|---|---|---|---|
| | | | | | | | diffraction | | | |
| | | | | | | | peak of | | | |
| | | | | Loose | | Average | X-ray | | | Final |
| | | Number | XXX | bulk | | particle | diffraction | Peak | Tempering | temperature |
| | Fat and/or oil | of carbon | triglyceride | density | Aspect | size | measurement | intensity | temperature/ | of cooling/ |
| | component | atoms x | (mass %) | (g/cm³) | ratio | (μm) | (Å) | ratio | time | time |
| Production | Tripalmitin | 16 | 89.7 | 0.2 | 2.0 | 119 | 4.6 | 0.90 | — | 50° C./ |
| Example 1 | | | | | | | | | | 12 hr. |
| Production | Hard palm stearin | 16 | 69.9 | 0.3 | 1.4 | 99 | 4.6 | 0.88 | — | 50° C./ |
| Example 2 | | | | | | | | | | 12 hr. |
| Production | Tripalmitin | 16 | 89.7 | 0.2 | 2.0 | 87 | 4.6 | 0.89 | 30° C./ | 50° C./ |
| Example 3 | | | | | | | | | 0.01 hr. | 2 hr. |
| Production | Tripalmitin | 16 | 89.7 | 0.2 | 2.0 | 92 | 4.6 | 0.89 | — | 60° C./ |
| Example 4 | | | | | | | | | | 2 hr. |
| Production | Tristearin | 18 | 99.6 | 0.2 | 2.0 | 30 | 4.6 | 0.93 | — | 50° C./ |
| Example 5 | | | | | | | | | | 12 hr. |
| Production | Tristearin | 18 | 96.0 | 0.2 | 2.0 | 31 | 4.6 | 0.88 | — | 55° C./ |
| Example 6 | | | | | | | | | | 12 hr. |
| Production | Highly hydrogenated | 18 | 79.1 | 0.2 | 1.6 | 54 | 4.6 | 0.89 | — | 55° C./ |
| Example 7 | rapeseed oil | | | | | | | | | 12 hr. |
| Production | Highly hydrogenated | 18 | 66.7 | 0.3 | 1.4 | 60 | 4.6 | 0.91 | — | 55° C./ |
| Example 8 | soybean oil | | | | | | | | | 12 hr. |
| Production | High-oleic highly | 18 | 83.9 | 0.2 | 1.6 | 48 | 4.6 | 0.89 | — | 55° C./ |
| Example 9 | hydrogenated | | | | | | | | | 12 hr. |
| | sunflower seed oil | | | | | | | | | |
| Production | Highly hydrogenated | 18 | 53.6 | 0.3 | 1.4 | 63 | 4.6 | 0.78 | — | 55° C./ |
| Example 10 | soybean oil, highly | | | | | | | | | 12 hr. |
| | hydrogenated palm oil | | | | | | | | | |
| Production | Tristearin | 18 | 96.0 | 0.2 | 2.0 | 36 | 4.6 | 0.88 | — | 70° C./ |
| Example 11 | | | | | | | | | | 12 hr. |
| Production | Highly hydrogenated | 18 | 79.1 | 0.2 | 1.6 | 50 | 4.6 | 0.90 | 50° C./ | 65° C./ |
| Example 12 | rapeseed oil | | | | | | | | 0.1 hr. | 6 hr. |
| Production | Highly hydrogenated | 18 | 79.1 | 0.2 | 1.6 | 52 | 4.6 | 0.89 | 40° C./ | 65° C./ |
| Example 13 | rapeseed oil | | | | | | | | 0.01 hr. | 2 hr. |
| Production | Highly hydrogenated | 18 | 79.1 | 0.2 | 1.6 | 60 | 4.6 | 0.89 | — | 65° C./ |
| Example 14 | rapeseed oil | | | | | | | | | 8 hr. |
| Production | Triarachidin | 20 | 99.5 | 0.2 | 2.0 | 42 | 4.6 | 0.92 | — | 72° C./ |
| Example 15 | | | | | | | | | | 12 hr. |
| Production | Tribehenin | 22 | 97.4 | 0.2 | 2.0 | 52 | 4.6 | 0.93 | — | 79° C./ |
| Example 16 | | | | | | | | | | 12 hr. |
| Production | Tripalmitin, Tristearin | 16, 18 | 93.8 | 0.2 | 1.6 | 74 | 4.6 | 0.90 | — | 55° C./ |
| Example 17 | | | | | | | | | | 16 hr. |
| Production | Hard palm stearin, | 16, 18 | 75.3 | 0.3 | 1.4 | 77 | 4.6 | 0.88 | — | 55° C./ |
| Example 18 | highly hydrogenated | | | | | | | | | 15 hr. |
| | rapeseed oil | | | | | | | | | |
| Comparative | Tripalmitin | 16 | 89.7 | — | — | — | 4.1 | 0.10 | — | 25° C/ |
| Production | | | | | | | | | | 4 hr. |
| Example 1 | | | | | | | | | | |
| Comparative | Hard palm stearin, | 16, 18 | 39.6 | — | — | — | 4.2 | 0.12 | — | 40° C./ |
| Production | highly hydrogenated | | | | | | | | | 12 hr. |
| Example 2 | palm oil | | | | | | | | | |
| Comparative | Highly hydrogenated | 18 | 79.1 | — | — | — | 4.1 | 0.11 | — | 40° C./ |
| Production | rapeseed oil | | | | | | | | | 3 hr. |
| Example 3 | | | | | | | | | | |
| Comparative | Highly hydrogenated | 18 | 39.7 | — | — | — | 4.2 | 0.12 | — | 55° C./ |
| Production | soybean oil, Highly | | | | | | | | | 12 hr. |
| Example 4 | extremely hydrogenated | | | | | | | | | |
| | palm oil | | | | | | | | | |

1. A 3-hydroxybutyric-acid-containing fat and/or oil composition comprising 3-hydroxybutyric acid and/or a salt of 3-hydroxybutyric acid, and
fat and/or oil particles,
the fat and/or oil particles having a plate-like shape and comprising a fat and/or oil component containing at least one type of XXX triglyceride having a Cₓ fatty acid residue X at positions 1 to 3 of glycerine, wherein x, which represents the number of carbon atoms, is an integer selected from 10 to 22, and
the fat and/or oil component comprising p-form fat and/or oil.

2. The composition according to claim 1, wherein the fat and/or oil particles are present in an amount of 0.5 to 100 parts by mass per 100 parts by mass of the total of the 3-hydroxybutyric acid and/or the salt of 3-hydroxybutyric acid.

3. The composition according to claim 1, wherein the salt of 3-hydroxybutyric acid is at least one member selected from the group consisting of sodium salt, potassium salt, calcium salt, and magnesium salt.

4. The composition according to claim 1, wherein the 3-hydroxybutyric acid and/or the salt of 3-hydroxybutyric acid has an R configuration.

5. A method for improving the deliquescence of 3-hydroxybutyric acid and/or a salt of 3-hydroxybutyric acid,
the method comprising mixing the 3-hydroxybutyric acid and/or the salt of 3-hydroxybutyric acid with fat and/or oil particles,
the fat and/or oil particles having a plate-like shape and containing at least one type of XXX triglyceride having a Cₓ fatty acid residue X at positions 1 to 3 of glycerin, wherein x, which represents the number of carbon atoms, is an integer selected from 10 to 22, and
the fat and/or oil component comprising p-form fat and/or oil.

6. A method for reducing the sour taste of 3-hydroxybutyric acid and/or a salt of 3-hydroxybutyric acid,
the method comprising mixing the 3-hydroxybutyric acid and/or the salt of 3-hydroxybutyric acid with fat and/or oil particles,
the fat and/oil particles having a plate-like shape and comprising a fat and/or oil component containing at least one type of XXX triglyceride having a Cₓ fatty acid residue X at positions 1 to 3 of glycerin, wherein x, which represents the number of carbon atoms, is an integer selected from 10 to 22, and
the fat and/or oil component comprising β-form fat and/or oil.
